(19) United States
(12) Patent Application Publication
(10) Pub. No.: US 2013/0030476 A1
(43) Pub. Date: Jan. 31, 2013

(54) BONE SCREW/DRIVER ASSEMBLY HAVING SURFACE ROUGHNESS

(71) Applicants: Daniel A. Shimko, Germantown, TN (US); Bryan Scott Berkebile, Walkerton, IN (US); Eric Crispen, Rochester, IN (US); Carlos Joel Lugo, Fort Wayne, IN (US)

(72) Inventors: Daniel A. Shimko, Germantown, TN (US); Bryan Scott Berkebile, Walkerton, IN (US); Eric Crispen, Rochester, IN (US); Carlos Joel Lugo, Fort Wayne, IN (US)

(73) Assignee: Warsaw Orthopedic, Inc., Warsaw, IN (US)

(21) Appl. No.: 13/193,839

(22) Filed: Jul. 29, 2011

(51) Int. Cl. A61B 17/86

(52) U.S. Cl. 606/308

(57) ABSTRACT

An assembly and method for attaching a medical device, or other structure, to a bone, according to which two projections are formed on one end of a driver that extend in two corresponding recesses, respectively, formed in the head of the screw to drivingly engage the driver and the screw.

### BACKGROUND

“Stab-n-grab” screw driver/screw interfaces are common in the medical device industry where one handed retrieval and delivery of screws is required. This functionality is achieved in several ways. One common way involves the insertion of a slightly tapered protrusion into either a tapered or straight void to create an interference fit. The result is the tapered protrusion is reversibly jammed into the hole allowing for a temporary connection.

One of the primary drawbacks of this kind of interference fit is that it requires very tight machine tolerances and a very smooth surface to work well. An example of this would be a morse taper on power drive connections in industrial applications. On large scales, these work well, but at small scales the physics breaks down with small surface abnormalities limiting contact at the interference point. Also, due to relatively high deformations at small scale, the repeatability of stab-n-grab operations is greatly diminished since the hole the tapered protrusion is pressed into will plastically deform making a second and third grab virtually impossible.

### BRIEF DESCRIPTION OF THE DRAWINGS

FIG. 1 is an isometric view of a bone screw/driver assembly according to an embodiment.

FIGS. 2 and 3 are isometric views of a bone screw/driver assembly according to two additional embodiments.

### DETAILED DESCRIPTION

In the present invention, it was surprisingly discovered that application of a heavy surface texture to the interface, either a projection a hole or both, significantly improves the strength of the reversible interference fit and allows the repeatability of the “stab-n-grab” operation.

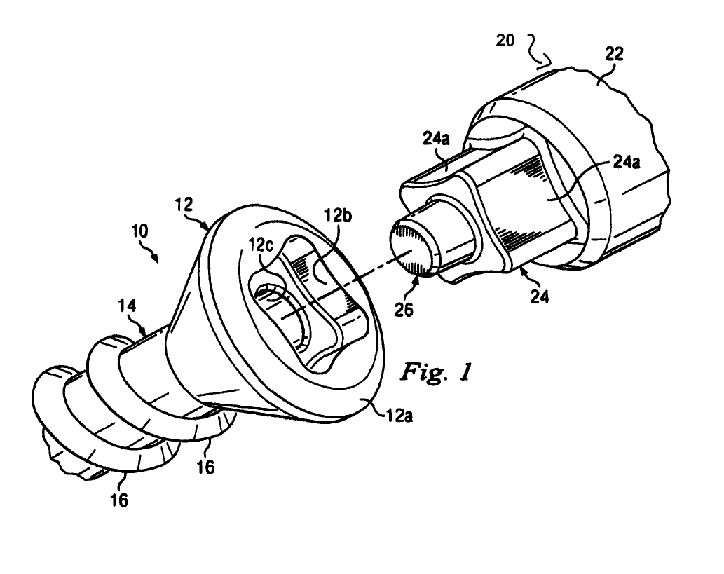

Referring to one embodiment of the invention as set forth in FIG. 1 of the drawings, the reference numeral 10 refers, in general, to a bone screw comprising a head 12 formed integrally with a shank 14. The head 12 is frusto-conical in cross-section, has a flat distal end surface 12a, and tapers inwardly in a direction from the distal end surface.

The shank 14 is substantially cylindrical in shape and has a helical thread 16 projecting from its outer surface. The screw is “self-tapping”, i.e. when it is driven into a bone, or other structure, by applying torque to the screw, the thread 16 functions to anchor the screw 10 in the bone. It is understood that the distal end portion (not shown) of the shank 14 could be tapered.

A driver is referred to, in general, by the reference numeral 20 and includes a cylindrical handle 22, shown partially. A polygonal projection 24 extends from one end of the handle and is formed integrally therewith. In one embodiment, and as shown in FIG. 1, the projection 24 has four walls 24a of substantially the same dimensions to form a substantially square cross-section. Each of the walls 24a is slightly curved to form a concave surface and the width of each wall is less than the diameter of the handle 22.

A cylindrical projection 26 extends from the distal end of the projection 24 and is formed integrally therewith. The diameter of the projection 24 is less than the width of each wall of the projection 24.

A recess 12b is formed in the head 12 of the screw 10, and extends from the flat surface 12a axially into the head. The cross-section of the recess 12b substantially corresponds to the cross-section of the projection 24, with the exception that its corresponding dimensions are slightly greater than the corresponding dimensions of the projection, so as to permit the projection to be inserted into the recess with minimal clearance. Alternatively, and in another embodiment, the polygonal projection 24 is in the shape of a hexagon, with the cross section of recess 12b having dimensions substantially corresponding to the cross section of the polygonal projection with the exception that its corresponding dimensions are slightly greater than the corresponding dimensions of the projection, so as to permit the projection to be inserted into the recess with minimal clearance.

A cylindrical recess, or counterbore, 12c, is also formed in the head 12 and extends from the bottom of the recess 12b further axially into the head. The cross-section of the recess 12c substantially corresponds to the cross-section of the cylindrical projection 26, with the dimensions of the respective cross-sections being such that the projection 26 fits into the recess 12c in an interference fit. (By the term “interference fit” here, and throughout this specification, applicants intend to mean the standard engineering definition of the term.) As mentioned previously, in the present invention it was surprisingly found that the application of a heavy texture to the projection (i.e cylindrical projection 26) significantly improves the strength of the reversible interference fit and allows for repeated use.

In use, the driver 20 is placed in operative engagement with the screw 10 by initially inserting the projection 26 through the recess 12b and into the recess 12c, and then rotating the driver 20 relative to the screw 10 or the screw relative to the driver until the projection 24 is in alignment with the recess 12b. The driver 20 is then moved further axially relative to the head 12 until the distal ends of the projections 24 and 26 engage the bottoms of the recesses 12b and 12c, respectively; and/or the flat surface of the end of the handle 22 engages the flat surface 12a of the head 12, to seat the projections in their respective recesses.

The interference fit afforded by the engagement of the projection 26 in the recess 12c enables the surgeon to connect the driver 20 to the screw 10 with one hand if necessary and then locate the end of the shank 14 at the bone. Torque is then applied to the handle 22, either manually or by an electrical device, which causes corresponding rotation of the driver 20 and therefore the screw 10. Axial force is applied to the screw 10 during the application of the torque, causing the screw to be driven into the bone in locking engagement therewith. If a medical device, or other structure, were to be attached to the bone by the screw 10, the screw would initially be inserted through an opening, or the like, in the device or structure before it is driven into the bone in the above manner.

Thus, the assembly of the present invention provides a secure locking engagement between the driver 20 and the screw 10. Also, the driver 20 can engage and drive the screw 10 relatively easily.

The inventors have surprisingly found that at a small scale (multiple millimeters) the amount of surface area contact using traditional smooth to smooth contact is exceedingly small, resulting in minimal interference strength. To overcome this lack of strength and increase the frequency [?] a strong interface can be created between the mating parts by adding a texture to the interface between the projection and the recess (for example 26 and 12C in FIG. 1).

Surface texture may be achieved on the relevant surface by a number of methods including, but not limited to glass bead blasting the surface or by shot peening methods. Alternatively, the texture may be created using modified cutting tools or custom tool paths during the manufacturing process itself. In one embodiment, the projection has a surface roughness (Rₐ) of from about 0.01 to 2. In another embodiment, the projection has a surface roughness (Rₐ) of from about 0.03 to about 1. In another embodiment, the projection has a surface roughness (Rₐ) of from about 0.1 to 0.8. One of ordinary skill in the art, using the disclosure of the present invention, would be able to determine the appropriate method for imparting the aforementioned surface roughness to the desired part surface.

In the present invention, the projection, the recess, or both could be appropriately treated to achieve the surface roughness described herein. In addition, the fit between the projection and the recess could be selected from: a tapered projection and a substantially cylindrical recess; a tapered projection and a tapered recess; or a cylindrical projection and a tapered recess.

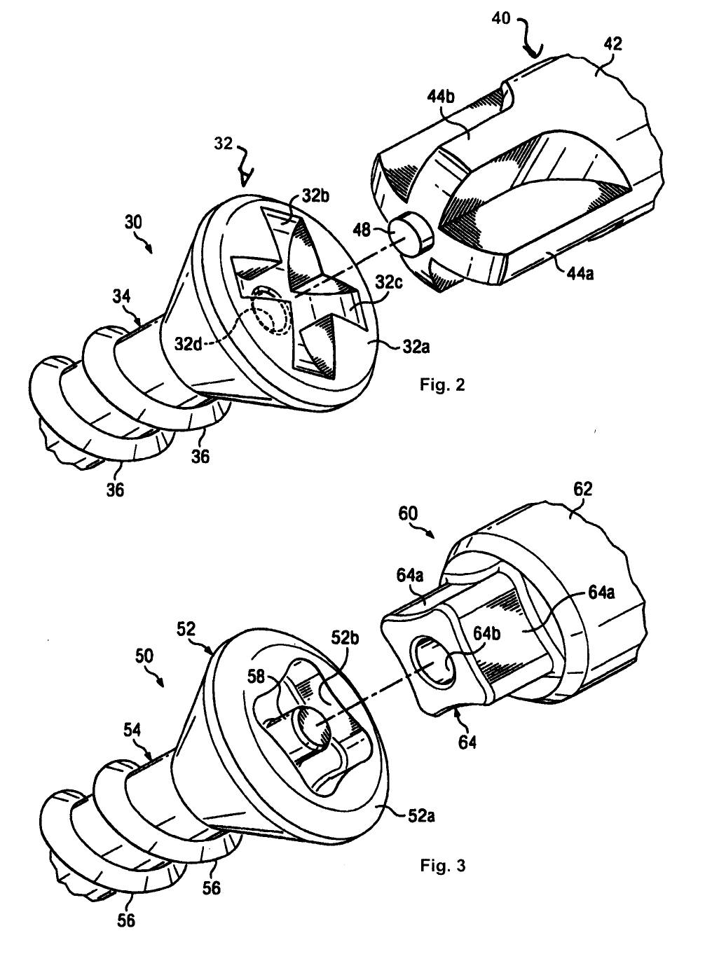

According to another embodiment of the inventions as set forth in FIG. 2, the projection, or pin 48 has a diameter less that the width of the each blade 44a and 44b. Again, the projection, the recess, or both have the surface roughness as described herein. The reference number 30 refers to a bone screw comprising a head 32 formed integrally with a shank 34. The head 32 is generally frusto-conical in cross-section and tapers inwardly in a direction from the distal end surface.

The shank 34 is substantially cylindrical in shape and has a helical thread projecting from its outer surface. The screw is “self-tapping”, i.e. when it is driven into bone or other structure by applying torque to the screw, the thread 36 functions to anchor the screw 30 into the bone. It is understood that the distal portion of the shank could be tapered.

A driver is referred to by reference numeral 40 and includes a cylindrical handle 42, shown partially. Two cross blades 44a and 44b are formed integrally with the handle and extend one end of the handle. The blades 44a and 44b extend at substantially ninety degrees to each other, and the outer surfaces of the bladed are curved to form convex surfaces. In other embodiments, the driver may be configure in other hexalobal shapes, including but not limited to hexagonal, polygonal, etc.

Two recesses or slots 32b and 32c extend from the end surface of 32a of the head 32 axially into the head and at ninety degrees to each other. The cross-section of the slots 32b and 32c substantially correspond to the cross-section of the blades 44a and 44b, with the exception that the dimensions of the slots are very slightly greater than the corresponding dimensions of the blades, so at to permit the blades to be inserted into the slots with minimal clearance.

A cylindrical recess, or counterbore, 32d, is formed in the head 32 and extends from the bottom of the centers of the slots 32b and 32c. The cross-section of the recess 32d substantially corresponds to the cross-section of the projection 48, with the dimensions of their respective cross-section being such that the projection fits into the recess in an interference fit.

In use, the driver 40 is placed in operative engagement with the screw 30 by initially inserting the projection 48 through the slots 32 b and 32c and into the recess 32d, and then rotating the driver 40 relative to the screw 30 until the blades 44a and 44b are in alignment with the slots 32b and 32c. The driver 40 is then moved further axially relative to the head 34 until the distal end of the projection 46 engages the bottom of the recess 32d, and the distal ends of the blades 44a and 44b engage the bottoms of the slots 32b and 32c, to seat the projections and the blades in their respective recesses.

The interference fit afforded by the engagement of the projection 48 into the recess 32d enables the surgeon to connect the drive 40 to the screw 30 with one hand if necessary and then locate the end of the shank 34 at the bone. Torque is then applied to the handle 42, either manually or by an electrical device, which causes corresponding rotation of the driver 40, and therefore the screw 30. Axial force is applied to the screw 30 during the application of the torque, causing the screw to be drive into the bone in locking engagement therewith. If a medical device or other structure were to be attached to the bone by the screw 50, the screw would initially be inserted through an opening in the device or structure before it is driven into the bone in the above manner. In this embodiment, and is described in reference to FIG. 1, the projection (48), the recess (32d) or both could have the modified surface reference as described in the present specification.

In a further embodiment of the invention, the projection may be incorporated into the screw and the recess may be incorporated into the drives. In this embodiment, the projection, the recess or both have the modified surface roughness as described herein. As shown in one embodiment of this invention that is shown in FIG. 3, the reference numeral 50 refers, in general, to a bone screw comprising a head 52 formed integrally with a shank 54. The head 52 is frusto-conical in cross-section, has a flat distal end surface 52a, and tapers inwardly in a direction from the distal end surface.

The shank 54 is substantially cylindrical in shape and has a helical thread 56 projecting from its outer surface. The screw is “self-tapping”, i.e. when it is driven into a bone, or other structure, by applying torque to the screw, the thread 56 functions to anchor the screw 50 in the bone. It is understood that the distal end portion (not shown) of the shank 54 could be tapered.

A recess 52b, having a substantially square cross-section, is formed in the head 52 and extends from the flat surface 52a axially into the head. A cylindrical projection 58 projects upwardly from the bottom of the center of the recess 52b. The distal end of the projection 58 extends approximately flush with the end surface 52a, and the diameter of the projection 58 is less than the corresponding dimensions of the recess 52b.

A driver is referred to, in general, by the reference numeral 60 and includes a cylindrical handle 62, shown partially. A polygonal projection 64 extends from one end of the handle and is formed integrally therewith. The projection 64 has four walls 64a of substantially the same dimension to form a substantially square cross-section. Each of the walls 64a is slightly curved to form a concave surface, and the width of each wall is less than the diameter of the handle 62. The cross-section of the projection 64 substantially corresponds to the cross-section of the recess 52b, with the exception that the corresponding dimensions of the projection are slightly less than the corresponding dimensions of the latter recess, so as to permit the projection to be inserted into the recess with minimal clearance.

A cylindrical recess, or counterbore, 64b is formed in the projection 64. The cross-section and depth of the recess 64b substantially corresponds to the cross-section of the cylindrical projection 58 of the screw 50, with the dimensions of the respective cross-sections being such that the projection 58 fits into the recess 64b in an interference fit.

In use, the driver 60 is placed in operative engagement with the screw 50 by initially rotating the driver 60 relative to the screw 50 until the projection 64 is in alignment with the recess 52b. The driver 60 is then moved further axially towards the head 52, causing the projection 64 to enter the recess 52b and the projection 58 to enter the recess 64b. The axial movement is continued until the end of the projection 64 engages the bottom of the recess 52b, and the end of the projection 58 engages the bottom of the recess 64b, to seat the projections into their respective recesses.

The interference fit afforded by the engagement of the projection 58 in the recess 64c enables the surgeon to connect the driver 60 to the screw 50 with one hand if necessary and then locate the end of the shank 54 at the bone. Torque is then applied to the handle 62, either manually or by an electrical device, which causes corresponding rotation of the driver 60, and therefore the screw 50. Axial force is applied to the screw 50 during the application of the torque, causing the screw to be driven into the bone in locking engagement therewith. If a medical device, or other structure, were to be attached to the bone by the screw 50, the screw would initially be inserted through an opening, or the like, in the device or structure before it is driven into the bone in the above manner.

Thus, the assembly of the present invention provides a secure locking engagement between the driver 60 and the screw 50. Also, the driver 60 can engage and drive the screw 50 relatively easily.

The present invention also includes packaging and kits where the parts described herein are sold together, separately or as part of a system.

### Variations

It is understood that variations may be made in the foregoing without departing from the invention and examples of some variations are as follows:
The design of the shank and/or the threads of each embodiment can be varied.
The dimensions of the projections, and their corresponding recesses in each embodiment can be varied.
The projections can have more or less than four walls.
The walls of the projections can be configured in a manner different than that shown in the drawing and described above. For example, one or more of the walls can be curved so as to form a convex surface.
The shape of the handles of each embodiment can be varied.
The assembly of the present invention can be used to fasten any type of medical device or structure to a bone.
The spatial references made above are for the purpose of illustration only and do not limit the specific orientation or location of the structure described above.

The preceding specific embodiments are illustrative of the practice of the invention. It is to be understood, therefore, that other expedients known to those skilled in the art or disclosed herein, may be employed without departing from the invention or the scope of the appended claims, as detailed above. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts a nail and a screw are equivalent structures.

What is claimed is:

1. An assembly for attaching a medical device, or other structure, to a bone, the assembly comprising:
a driver comprising:
a body member;
a first projection extending from an end of the body member, the first projection having a polygonal shape and
a second projection extending from the first projection; and
a screw comprising:
a head,
a first recess formed in the head and having a cross-section substantially corresponding to the cross-section of the first projection, and
a second recess formed in the head and having a cross-section substantially corresponding to the cross-section of the second projection;
so that when the projections are seated in their respective recesses, the driver and the screw are drivingly connected and wherein the second projection, the second recess, or both, has a surface roughness of from about 0.01 to about 2.

2. The assembly of claim 1 wherein the second projection, the second recess, or both, has a surface roughness for from about 0.03 to about 1.

3. The assembly of claim 1 wherein the second projection, the second recess, or both, has a surface roughness of from about 0.1 to about 0.8.

4. The assembly of claim 1 wherein the second projection is cylindrical.

5. The assembly of claim 1 wherein the first projection is hexagonal.

6. The assembly of claim 1 wherein the second projection is conical.

7. The assembly of claim 1 wherein the second recess is conical.

8. The assembly of claim 1 wherein the second projection is tapered.

9. The assembly of claim 1, wherein the second recess is tapered.

10. The assembly of claim 1, wherein the second projection is cylindrical and the second recess is tapered.

11. A method of improving the strength of a reversible interference fit in an assembly for attaching a medical device, or other structure, to a bone, the assembly comprising:
a driver comprising:
a body member;
a first projection extending from an end of the body member, the first projection having a polygonal shape and
a second projection extending from the first projection; and
a screw comprising:
a head,
a first recess formed in the head and having a cross-section substantially corresponding to the cross-section of the first projection, and
a second recess formed in the head and having a cross-section substantially corresponding to the cross-section of the second projection;
the method comprising adding surface texture to the second projection, the second recess or both, and wherein the second projection, the second recess or both have a surface roughness of from about 0.01 to about 2.0 after application of the surface texture.

12. The method of claim 11, wherein the method of adding surface texture comprises glass bead blasting a surface of the second projection, the second recess, or both.

13. The method of claim 11 wherein the method of adding surface texture comprises shot peening a surface of the second projection, the second recess, or both.

14. The method of claim 11 wherein the second projection, the second recess or both have a surface roughness of from about 0.03 to about 1 after application of the surface texture.

15. The method of claim 11 wherein the second projection, the second recess or both have a surface roughness of from about 0.1 to about 0.8

16. A kit comprising a driver and a screw,
the driver comprising:
a body member;
a first projection extending from an end of the body member, the first projection having a polygonal shape and
a second projection extending from the first projection;
the screw comprising
a head,
a first recess formed in the head and having a cross-section substantially corresponding to the cross-section of the first projection, and
a second recess formed in the head and having a cross-section substantially corresponding to the cross-section of the second projection;
wherein the second projection of the driver or the second projection of the screw, or both, has a surface roughness of from about 0.01 to about 2.

17. The kit of claim 16, wherein the second projection of the driver or the second projection of the screw, or both, have a surface roughness for from about 0.03 to about 1.

18. The kit of claim 16, wherein the second projection of the driver or the second projection of the screw, or both, have a surface roughness of from about 0.1 to about 0.8.

19. The kit of claim 16 wherein the first projection is hexagonal.

20. The kit of claim 16, wherein the second projection is tapered.
